(19) United States
(12) Patent Application Publication
(10) Pub. No.: US 2008/0103103 A1
(43) Pub. Date: May 1, 2008

(54) Reagents and methods to treat ocular diseases and infection

(76) Inventors: Bahram Memarzadeh, San Carlos, CA (US); Lyle Bowman, Pleasanton, CA (US)

Correspondence Address: MCDERMOTT, WILL & EMERY, 4370 LA JOLLA VILLAGE DRIVE, SUITE 700, SAN DIEGO, CA, 92122, US

(21) Appl. No.: 11/590,617

(22) Filed: Oct. 30, 2006

(51) Int. Cl. A61K 31/7048; A61K 31/353; A61K 31/12; A61K 31/05

(52) U.S. Cl. 514/27; 514/456; 514/454; 514/733; 514/678

(57) ABSTRACT

The invention provides compositions and methods of treating an ocular condition by administering to an eye of a patient having an ocular condition an effective amount of a catechin or polyphenol. The compositions and methods can be used to treat ocular conditions such as ocular infection, ocular inflammation, ocular cancer or benign eye tumors.

The present invention relates generally to the field of medicine and more specifically to pharmaceutical compositions and method for topical application for treating or preventing viral infection, inflammation, and cancer in the eye.

### BACKGROUND OF THE INVENTION

The conjunctiva is the thin, clear membrane over the white part of the eye, and it also lines the eyelids. As with any mucous membrane, infectious agents can adhere to the conjunctiva and overwhelm normal defense mechanism. Inflammation of this membrane is called conjunctivitis. Conjunctivitis, also known as “pink eye,” is one of the most common nontraumatic eye complaints. Causes of conjunctivitis include allergic, viral, bacterial, chlamydial, parasitic and chemical agents. The causes of conjunctivitis can vary seasonally, with some causes increasing or decreasing in different seasons.

Conjunctivitis can be highly contagious, especially in environments with close human contact. Conjunctivitis outbreaks in schools or daycare facilities can result in the spread to many students or young children. Additionally, close confinement such as found in the military can also be an environment in which conjunctivitis can occur and spread rapidly. Other types of environments in which conjunctivitis can occur and readily spread include swimming pools, campgrounds, hotels, hospitals, nursing homes, offices, or other environments in which close human contact is common. Although conjunctivitis is generally self-limiting, conjunctivitis can progress to increasingly sever and sight-threatening infections, depending on the immune state of the patient and the etiology.

Due to the ease with which conjunctivitis can spread and the impact it can have on lost work hours in business and the military as well as lost student hours, as well as the possibility that conjunctivitis can lead to more severe, sight threatening infections, it is important that effective methods are used to treat conjunctivitis. Antibiotics are often used to treat conjunctivitis but are ineffective or poorly effective for treating viral conjunctivitis. For viral conjunctivitis, treatment is usually limited to symptomatic therapy, much as one would treat the common cold. Vasoconstrictor and antihistamine combinations in eye-drop form may be very helpful in relieving symptoms. In cases in which subepithelial infiltrates develop and affect vision, steroids may sometimes be recommended to control symptoms and speed recovery. However, it is quite possible that once the steroids are discontinued, the disease may continue to run its course. Furthermore, long-term steroid use may be associated with development of cataracts or glaucoma.

Thus, there exists a need for effective treatments of conjunctivitis or other eye conditions. The present invention satisfies this need and provides related advantages as well.

### SUMMARY OF INVENTION

The invention provides compositions and methods of treating an ocular condition by administering to an eye of a patient having an ocular condition an effective amount of a catechin or polyphenol. The compositions and methods can be used to treat ocular conditions such as ocular infection, ocular inflammation, ocular cancer or benign eye tumors.

### BRIEF DESCRIPTION OF THE DRAWINGS

FIG. 1 shows the structure of epigallocatechin gallate (EGCG).

### DETAILED DESCRIPTION OF THE INVENTION

The present invention provides compositions and methods for treating conjunctivitis and other eye conditions. The invention is based on the use of epigallocatechin gallate (EGCG) to treat conjunctivitis or other eye conditions such as cancer of the eye or inflammation of the eye and/or surrounding tissues. Despite the lack of complete knowledge about eye disease, there are several known causes for the inflammation, infection, and cancer of the eye. The infection of the eye is also known as “pink eye” and is manifested as epidemics in school children, military personnel or in the general population. The present invention relates to the use of pharmaceutical preparations to ameliorate signs or symptoms associated with eye conditions such as conjunctivitis, inflammation of the eye or eye cancer, for example, by shortening the duration of conjunctivitis or eye inflammation or reducing the severity of eye cancer.

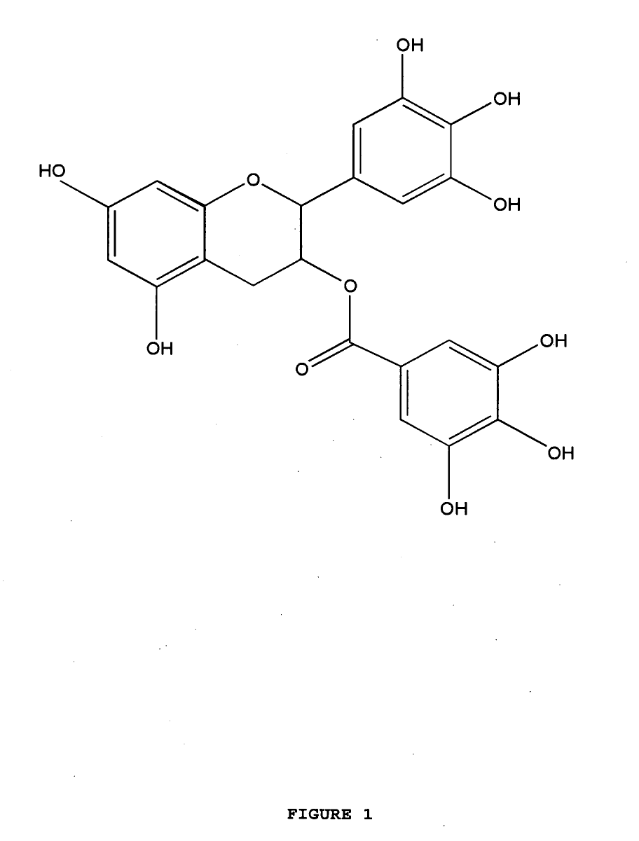

Compositions and methods of the invention are based on the use of epigallocatechin gallate. Epigallocatechin gallate (EGCG) belongs to the family of catechins and is a member of the chemical class known as polyphenols (see FIG. 1). EGCG is a potent antioxidant found in black tea or Chinese green tea, from which it can be extracted. EGCG is estimated to be 10 to 50% of the total green tea catechins, which consists of epicatechin (EC), epicatechin gallate (EC G), epigallocatechin (EGC), epigallocatechin gallate (EGCG), gallocatechin gallate (GCG). While exemplified herein with EGCG, it us understood that other catechins or polyphenols can be used in compositions and methods of the invention, including but not limited to, EC, ECG, EGC, GCG, and the like. Other exemplary catechins or polyphenols include apigenin, anthocyanins, aurones, chalcones, isoflavones, proanthocyanidins, astringin, coumarins, stilbenes, xanthones, and the like. Although exemplified herein with EGCG, it is understood that any of the catechins or polyphenols, alone or in combination, can be used in compositions and methods of the invention. Catechins or polyphenols can be extracted from natural sources such as green or black tea. Catechins and other polyphenols are also available commercially (see, for example, Sigma-Aldrich, St. Louis Mo., and LKT Laboratories, St. Paul Minn.). Alternatively, catechins or polyphenols can be synthesized using well known methods of chemical synthesis. Thus, the invention provides a composition comprising a catechin or polyphenol, as disclosed herein. In a particular embodiment, the invention provides a pharmaceutical composition containing a catechin or polyphenol and a pharmaceutically acceptable carrier.

The chemical formula for EGCG is C₂₂H₁₈O₁₁ and is also referred to as (2R,3R)-2-(3,4,5-Trihydroxyphenyl)-3,4-dihydro-1(2H)-benzopyran-3,5,7-triol 3-(3,4,5-trihydroxybenzoate). It has a molecular weight of 458.37 and is commercially available as highly purified raw material.

The invention provides methods of treating an ocular condition. In one embodiment, the invention provides a method of treating an ocular infection by administering to an eye of a patient having an ocular infection an effective amount of (−)-epigallocatechin gallate (EGCG). In another embodiment, the invention provides a method of ameliorating a sign or symptom associated with an ocular infection by administering to an eye of a patient having an ocular infection an effective amount of (−)-epigallocatechin gallate (EGCG).

Conjunctivitis, also known as pink eye, is caused by a variety of agents, including allergic, viral, bacterial, chlamydial, parasitic and chemical agents. Conjunctivitis caused by infectious agents is highly contagious. In a particular embodiment of the invention, a method is provided to treat a viral ocular infection, for example, an adenoviral ocular infection. A method is also provided for prophylactic treatment to prevent an infection, for example, in prior to or after an ocular surgical procedure.

Viral conjunctivitis can be caused by a variety of viruses. Adenovirus is the most common cause of viral conjunctivitis and varies seasonally, most frequently found in the fall. Other exemplary viral etiological agents include herpes simplex virus (HSV), varicella-zoster virus (VZV), picornoviruses such as enterovirus 70 and Coxsackie A24 virus, poxvirus such as molluscum contagiosum and vaccinia, and human immunodeficiency virus (HIV).

Methods of the invention can be used to ameliorate a sign or symptom associated with an ocular infection such as conjunctivitis. Signs or symptoms associated with ocular conjunctivitis include, for example, eyelids sticking together, itching and burning, a gritty foreign-body sensation in the eye, and discharge. Bacterial conjunctivitis is characterized by acute onset, minimal pain, occasional pruritis (itching). Ocular surface disease, such as keratitis sicca, trichiasis, or chronic blepharitis, predisposes the patient to bacterial conjunctivitis. Staphylococcal and streptococcal species are the most common pathogens for bacterial conjunctivitis.

Viral conjunctivitis is characterized by acute or subacute onset and minimal pain level. Pruritus is common, and a clear, watery discharge is typical. Occasionally, severe photophobia and foreign-body sensation occurs, usually caused by adenovirus (epidemic keratoconjunctivitis (EKC)), when associated with keratitis. Chlamydial conjunctivitis is characterized by chronic onset, minimal pain level, occasional pruritus, and is often associated with a history of sexually transmitted disease. Allergic conjunctivitis is characterized by acute or subacute onset and no pain. Pruritus is extremely common. Clear, watery discharge is typical with or without a moderate amount of mucous production. An aggressive form of allergic conjunctivitis is vernal conjunctivitis in children and atopic conjunctivitis in adults. Vernal disease often is associated with shield corneal ulcers. Perilimbal accumulation of eosinophils typifies vernal disease. Vernal keratoconjunctivitis (VKC), usually affecting young boys, tends to be bilateral and occurs in warm weather. VKC is presumed to be a hypersensitivity to exogenous antigens and may be associated with or accompanied by keratoconus.

Giant papillary conjunctivitis resembles vernal disease. This condition occurs mainly in contact lens wearers who develop a syndrome of excessive pruritus, mucous production, and increasing intolerance to contact use. The giant papillae are predominantly on the upper palpebral conjunctiva and can be seen only on lid eversion.

Further exemplary signs and symptoms associated with viral conjunctivitis include, for example, discharge, irritation, red eye, increased tearing, eye pain and facial pain, itching of the eye (pruritis), gritty feeling in the eyes, blurred vision, sensitivity to light, and crusts that form on the eyelid overnight. Infection generally begins in one eye, but can spread to the other eye. There is generally less discharge in viral conjunctivitis than in bacterial conjunctivitis.

Adenovirus is one of the most common causes of viral conjunctivitis. Adenoviruses are nonenveloped, double-stranded DNA viruses. There are 49 immunologically distinct types, with 6 subgenera, A to F, that can cause human infections. Adenoviruses are unusually stable to chemical or physical agents and adverse pH conditions, allowing for prolonged survival outside the body. Epidemic keratoconjunctivitis is associated with adenovirus serotypes 8, 19 and 37. Epidemics of febrile disease with conjunctivitis are associated with waterborne transmission of some adenovirus types.

Various agents can be used to inhibit or inactivate adenovirus, although adenoviruses are unusually stable to chemical or physical agents and adverse pH conditions, allowing for prolonged survival outside the body. Adenovirus can remain active in alcohol solutions and in pH as high as pH 9 or as low as pH 5. Some surfactants can completely denature adenovirus but can be harmful to tissue. The stability of adenovirus under fairly extreme conditions also means that these agents that can inhibit or inactivate adenoviruses can be too harsh to apply for treatment of an infection, particularly to the eye.

Anti-adenovirus agents used for ocular applications have some activity. For example, 3% gancicolovir has been tested with very minor activity against adenovirus. Animal studies with 0.5% cidofovir showed some positive results, but 0.2% cidofovir failed in clinical trials. Other anti-viral agents for treating other viral etiological agents for conjunctivitis have been tested for the respective infections. For example, anti HIV and HSV compounds have been tested but not found to be effective alone. Acyclovir and famciclovir are effective against HSV early onset but not adenovirus.

As disclosed herein, EGCG is an effective agent to treat conjunctivitis, in particular viral conjunctivitis caused by adenovirus. As disclosed herein, EGCG was found to be active against adenovirus in vitro, greatly reducing the viral load. In vitro studies show the inhibitory effects and therapeutic index of greater than 20. A concentration of 0.005% is effective for in vitro cultures (see Examples).

Compositions of the invention are generally formulated for ocular administration, particularly in formulations suitable for ophthalmic administration such as by instillation. Compositions of the invention can be formulated in ophthalmic solutions, suspensions, ointments, and the like, using well known methods (see, for example, Remington's Pharmaceutical Sciences 18th ed., Gennaro, ed., Mack Publishing Company, Easton, Pa. (1990)). Other suitable modes of administration include ocular inserts, intraocular administration, packs, intracameral injections, iontophoresis, subjconjunctival injections, retrobulbar injections, and the like.

The compositions of the invention are formulated in suitable vehicles that include buffers, salts, and pharmaceutical carriers. For example, a composition can be formulated in a sterile isotonic solution, for example, saline or boric acid solutions. Such formulations suitable for ophthalmic applications discussed in more detail below and are well known to those skilled in the art (see Remington's Pharmaceutical Sciences, supra, Chapter 86).

The compositions of the invention can further include ophthalmic preservatives for inhibiting microbial growth. Exemplary ophthalmic preservatives include, but are not limited to, quaternary ammonium compounds, organic mercurials, parahydroxy benzoates, chlorobutanol, aromatic alcohols, and the like. Other additives can also be included, for example, antioxidants such as sodium bisulfite or metabisulfite, ascorbic acid, acetylcysteine, and the like. Surfactants can also be included in a composition of the invention, for example, ionic, non-ionic, zwitterionic, or amphiphilic surfactants. In a composition of the invention, EGCG or other catechins or polyphenols can also be administered with a penetration-enhancing agent. Exemplary penetration-enhancing agents include, but are not limited to, sodium citrate, dodecyl maltoside, sucrose monolaurate, polydecanol, sodium dodecyl sulfate, 3[(3-Cholamidopropyl)dimethylammonio]-propanesulfonic acid (CHAPS) and related compounds, polypeptides, and the like (see, for example, U.S. publication 2005/0085427).

According to one aspect of the present invention, a pharmaceutical preparation of EGCG or other catechin or polyphenol is made as a solution of EGCG or other catechin or polyphenol in a buffer to contain soluble amounts of EGCG or other catechin or polyphenol up to about 5% and in particular up to about 0.5% to be applied to a virus infected eye. For example, a composition of the invention can contain about 5%, about 4%, about 3%, about 2%, about 1.5%, about 1.2%, about 1%, about 0.9%, about 0.8%, about 0.7%, about 0.6%, about 0.5%, about 0.4%, about 0.3%, about 0.2%, about 0.1%, about 0.05% or even lower of EGCG or other catechin or polyphenol. Lower concentrations of catechins or polyphenols can be particularly useful, for example, in combination with other components or agents such as antiviral agents.

According to another aspect of the present invention, EGCG or other catechins or polyphenols can be dissolved in a pharmaceutically accepted ointment or oil to its limit of solubility, and in particular to about 10%, and applied to the virus infected eye of a patient. For example, a composition of the invention in an ointment can contain about 10%, about 9%, about 8%, about 7%, about 6%, about 5%, about 4%, about 3%, about 2%, about 1%, about 0.5%, about 0.4%, about 0.3%, about 0.2%, about 0.1%, about 0.05% or even lower concentration EGCG or other catechins or polyphenols.

According to yet another aspect of the present invention, catechins or polyphenols such as EGCG can be dissolved in a pharmaceutically accepted buffer in the presence of a pharmaceutical surfactant, such as an ionic, non-ionic, zwitterionic, or amphiphilic surfactant, to its limit of solubility, and in particular to about 10%, and applied to the virus infected eye of a patient. For example, a composition of the invention in a surfactant can contain about 10%, about 9%, about 8%, about 7%, about 6%, about 5%, about 4%, about 3%, about 2%, about 1%, about 0.5%, about 0.4%, about 0.3%, about 0.2%, about 0.1%, about 0.05% or even lower concentration of EGCG or other catechins or polyphenols.

The invention also provides methods for treating an eye infection using catechins or polyphenols such as EGCG administered with an antimicrobial agent. The antimicrobial agent can be an antiviral agent, an antibacterial agent, or an anti-fungal agent.

In a particular embodiment of the invention, catechins or polyphenols such as EGCG can be formulated with another antiviral agent and applied to the virus-infected eye. Any of one or more of a variety of antiviral agents can be combined with catechins or polyphenols such as EGCG in a composition of the invention. Exemplary antiviral agents include, but are not limited to, cidofovir, ganciclovir, acyclovir, famcicolovir, and the like. A particularly useful antiviral agent for combining with catechins or polyphenols such as EGCG for use in methods of the invention is cidofovir. Other exemplary antiviral agents include, but are not limited to, abacavir, acyclovir, adefovir, amantidine, ampligen, amprenavir, atevirdine, 3′-azido-2′,3′-dideoxyuridine (AZdU(CS-87)), capsaicin, cidofovir, delavirdine, didanosine, efaviren, emtricitabine, enfuvirtide, entecavir, enviroxime, famciclovir, fomivirsen, foscamet, ganciclovir, hydroxyurea, indinavir, interferon alfa, lamivudine, lovirdine, nelfinavir, nevirapine, oseltamivir, penciclovir, pleconaril, podofilox, podophyllin, ribavirin, rimantidine, ritonavir, saquinavir, stavudine, trifluridine, valacyclovir, vidarabine, zalcitabine, zanamivir, zidovudine, and the like. Thus, the invention provides a pharmaceutical composition containing one or more catechins or polyphenols and one or more antiviral agents.

In yet another embodiment of the invention, a composition of the invention can contain catechins or polyphenols such as EGCG formulated in combination with an antibiotic. Such a combination is particularly useful for treating infections of the eye caused by a virus, bacteria, or both. Exemplary antibiotics are disclosed herein below.

The invention additionally provides a method of treating ocular inflammation by administering to an eye of a patient having ocular inflammation an effective amount of a catechin or polyphenol such as (−)-epigallocatechin gallate (EGCG). The invention further provides a method of ameliorating a sign or symptom associated with ocular inflammation by administering to an eye of a patient having ocular inflammation an effective amount of a catechin or polyphenol such as (−)-epigallocatechin gallate (EGCG). Thus, the compositions of the invention can additionally be used to treat ocular inflammation, for example, iritis, intermediate ocular inflammation, retinitis, choroiditis, uveitis, episcleritis, scleritis, keratoconjunctivitis sicca, optic neuritis, Graves' orbitopathy, and the like.

The invention further provides a method of treating an ocular cancer by administering to an eye of a patient having an ocular cancer an effective amount of a catechin or polyphenol such as (−)-epigallocatechin gallate (EGCG). The catechin or polyphenol such as EGCG can be formulated with one or more chemotherapeutic agents for treatment of viral infections or cancer of the eye. Such a combination can be particularly useful for treating late-stage cancer of the eye or advanced viral infection of the eye. Exemplary ocular cancers include, for example, retinoblastoma, rhabdomyosarcoma, choroidal hemangioma, choroidal melanoma, choroidal metastasis, choroidal nevus, conjunctival tumors, eylid, tumors, iris tumors, lymphoma or leukemia, melanocyotoma, and orbital tumors.

Exemplary chemotherapeutic agents include, but are not limited to, alkylating agents, anthracycline, antineoplastic, azathioprine, bleomycin, bortezomib, bryostatin, busulfan, capecitabine, carboplatin, chlorambucil, cisplatin, clofarabine, cyclophosphamide, cytarabine, dacarbazine, daunorubicin, discodermolide, docetaxel, epirubicin, estramustine, etoposide, floxuridine, fludarabine, fluorouracil, gemcitabine, idarubicin, ifosfamide, irinotecan, lenalidomide, mechlorethamine, melphalan, mercaptopurine, methotrexate, mitomycin, mitoxantrone, nelarabine, oxaliplatin, paclitaxel, pemetrexed, pentostatin, procarbazine, streptozotocin, taxane, temozolomide, topotecan, valrubicin, vinblastine, vincristine, vinorelbine, and the like. Thus, the invention provides a pharmaceutical composition containing one or more catechins or polyphenols and one or more chemotherapeutic agents.

The invention also provides a method of treating a benign eye tumor by administering to an eye of a patient having a benign eye tumor an effective amount a catechin or polyphenol such as of (−)-epigallocatechin gallate (EGCG). Such methods can be useful in inhibiting conversion of a benign growth to a cancerous growth. Exemplary benign tumors include inflammatory pseudotumor of the orbit.

The compositions of the invention are administered in an effective amount to ameliorate a sign or symptom associated the condition being treated, for example, ocular infection, ocular inflammation, ocular cancer, benign eye tumor, and the like. Methods for topical treatment or prevention of ocular infections or other eye conditions are well known in the art (see U.S. Pat. Nos. 6,239,113 and 6,569,443, each of which is incorporated herein by reference). Where the dosing regimen includes a series of applications, it is possible that one or more of the earlier applications will not achieve an effective concentration in the ocular tissue, but that a later application in the regimen will achieve an effective concentration. This is contemplated as being within the scope of topically applying an composition of the invention in an effective amount. However, generally a single application, such as consisting of one or two drops, provides a therapeutically effective concentration of a composition of the invention within a tissue of the eye. Although dependent on the amount and form of the ophthalmic composition, a single application will typically provide a therapeutically effective amount of the composition of the invention containing a catechin or polyphenol such as EGCG within a tissue of the eye for at least 8, at least 12, or at least 18 hours.

The topical application of a composition of the invention can be used to treat or prevent a variety of conditions associated with ocular infection. For example, conditions of the lids including blepharitis, blepharconjunctivitis, meibomianitis, acute or chronic hordeolum, chalazion, dacryocystitis, dacryoadenities, and acne rosacea; conditions of the conjunctiva including conjunctivitis, ophthalmia neonatorum, and trachoma; conditions of the cornea including corneal ulcers, superficial and interstitial keratitis, keratoconjunctivitis, foreign bodies, and post operative infections; and conditions of the anterior chamber and uvea including endophthalmitis, infectious uveitis, and post operative infections, are a few of the tissues and conditions that can be treated by topical application of a composition of the invention containing a catechin or polyphenol such as EGCG. The prophylactic treatment to prevent or inhibit infection includes pre-operative treatment prior to surgery or post-operative treatment after surgery as well as other suspected infectious conditions or contact. Examples of prophylaxis situations include treatment prior to surgical procedures such as blepharoplasty, removal of chalazia, tarsorrhapy, procedures for the canualiculi and lacrimal drainage system and other operative procedures involving the lids and lacrimal apparatus; conjunctival surgery including removal of ptyregia, pingueculae and tumors, conjunctival transplantation, traumatic lesions such as cuts, burns and abrasions, and conjunctival flaps; corneal surgery including removal of foreign bodies, keratotomy, and corneal transplants; refractive surgery including photorefractive procedures; glaucoma surgery including filtering blebs; paracentesis of the anterior chamber; iridectomy; cataract surgery; retinal surgery; and procedures involving the extra-ocular muscles. The treatment, inhibition or prevention of ophthalmia neonatorum is also included.

More generally, the compositions of the invention containing a catechin or polyphenol such as EGCG, alone or in combination with other medicaments, can be used to treat ocular infections caused by a variety of viruses, bacteria or parasites, as disclosed herein, including but not limited to one or more of the following organisms: Staphylococcus including Staphylococcus aureus and Staphylococcus epidermidis; Streptococcus including Streptococcus pneumoniae and Streptococcus pyogenes as well as Streptococci of Groups C, F, and G and Viridans group of Streptococci; Haemophilus influenza including biotype III (H. aegyptius); Haemophilus ducreyi; Moraxella catarrhalis; Neisseria including Neisseria gonorrhoeae and Neisseria meningitidis; Chlamydia including Chlamydia trachomatis, Chlamydia psittaci, and Chlamydia pneumoniae; Mycobacterium including Mycobacterium tuberculosis and Mycobacterium avium-intracellular complex as well as atypical mycobacterium including M. marinum, M. fortuitm, and M. chelonae; Bordetella pertussis; Campylobacter jejuni; Legionella pneumophila; Bacteroides bivius; Clostridium perfringens; Peptostreptococcus species; Borrelia burgdorferi; Mycoplasma pneumoniae, Treponema pallidum; Ureaplasma urealyticum; toxoplasma; malaria; and nosema.

A composition of the invention containing a catechin or polyphenol such as EGCG is applied to the exterior surface of the eye, usually in an ophthalmically acceptable composition which comprises an ophthalmically acceptable carrier and a catechin or polyphenol such as EGCG. The “ophthalmically acceptable carrier” is used in a broad sense and includes any material or composition that can contain and release the a catechin or polyphenol such as EGCG composition and that is compatible with the eye. Typically the ophthalmically acceptable carrier is water or an aqueous solution or suspension, but also includes oils such as those used to make ointments and polymer matrices such as used in ocular inserts. An aqueous solution of a composition containing a catechin or polyphenol such as EGCG can be formed and used for topical application. In addition, an aqueous suspension can be formed. Ointments and solid dosage forms can also be used as delivery compositions as are well known in the art. The concentration of a catechin or polyphenol such as EGCG present in the ophthalmic composition depends upon the dosage form, the release rate, the dosing regimen, and the location and type of infection.

The fluid ophthalmic compositions of the present invention, including both ointments and suspensions, have a viscosity that is suited for the selected route of administration. A viscosity in the range of from about 1,000 to 30,000 centipoise is useful for a drop. About 30,000 to about 100,000 centipoise is an advantageous viscosity range for ophthalmic administration in ribbon form. The viscosity can be controlled in many ways known to those skilled in the art.

The ophthalmic compositions can contain one or more of the following: surfactants, adjuvants including additional medicaments, buffers, antioxidants, tonicity adjusters, preservatives, thickeners or viscosity modifiers, and the like. Additives in the formulation can desirably include sodium chloride, ethylenediamine tetraacetic acid (EDTA) and/or benzalkonium chloride (BAK), sorbic acid, methyl paraben, propyl paraben, chlorhexidine, and sodium perborate.

As discussed above, a composition of the invention can include components in combination with a catechin or polyphenol such as EGCG such as additional medicaments. A composition comprising a composition of the invention containing a catechin or polyphenol such as EGCG, an additional medicament, and an ophthalmically acceptable carrier can advantageously simplify administration and allow for treating or preventing multiple conditions or symptoms simultaneously. The “additional medicaments,” which can be present in any of the ophthalmic compositional forms described herein including fluid and solid forms, are pharmaceutically active compounds having efficacy in ocular application and which are compatible for use with a catechin or polyphenol such as EGCG and with the eye. The additional medicaments can include antibiotics, antivirals, antifungals, anesthetics, anti-inflammatory agents including steroidal and non-steroidal anti-inflammatories, anti-allergic agents, chemotherapeutic agents, and the like. Examples of suitable medicaments include aminoglycosides such as amikacin, gentamycin, tobramycin, streptomycin, netilmycin, and kanamycin; fluoroquinolones such as ciprofloxacin, norfloxacin, ofloxacin, trovafloxacin, lomefloxacin, levofloxacin, and enoxacin; naphthyridine; sulfonamides; polymyxin; chloramphenicol; neomycin; paramomomycin; colistimethate; bacitracin; vancomycin; tetracyclines; rifampin and its derivatives (“rifampins”); cycloserine; beta-lactams; cephalosporins; amphotericins; fluconazole; flucytosine; natamycin; miconazole; ketoconazole; corticosteroids; diclofenac; flurbiprofen; ketorolac; suprofen; comolyn; lodoxamide; levocabastin; naphazoling; antazoline; and pheniramimane. The additional medicament can also include an azalide antibiotic (see U.S. Pat. Nos. 6,239,113 and 6,569,443). These other medicaments are generally present in a pharmaceutically effective amount as is understood by those of ordinary skill in the art. These amounts are generally within the range of from about 0.01 to about 5%, more typically about 0.1 to about 2%, for fluid compositions and from about 0.5 to about 50% for solid dosage forms.

The aqueous ophthalmic compositions, solutions or suspensions, for use in the present invention use water which has no physiologically or ophthalmically harmful constituents. Typically purified or deionized water is used. The pH is adjusted by adding physiologically and ophthalmically acceptable pH adjusting acids, bases or buffers to within the range of about 5.0 to about 8.5. Examples of acids include acetic, boric, citric, lactic, phosphoric, hydrochloric, and the like, and examples of bases include sodium hydroxide, sodium phosphate, sodium borate, sodium citrate, sodium acetate, sodium lactate, tromethamine, THAM (trishydroxymethylamino-methane), and the like. Salts and buffers include citrate/dextrose, sodium bicarbonate, ammonium chloride and mixtures of the aforementioned acids and bases.

The osmotic pressure (π) of the aqueous ophthalmic composition is generally from about 10 milliosmolar (mOsM) to about 400 mOsM, in particular from about 260 to about 340 mOsM. If necessary, the osmotic pressure can be adjusted by using appropriate amounts of physiologically and ophthalmically acceptable salts or excipients. Sodium chloride is generally used to approximate physiologic fluid, and amounts of sodium chloride ranging from about 0.01% to about 1% by weight, and in particular from about 0.05% to about 0.45% by weight, based on the total weight of the composition, are typically used. Equivalent amounts of one or more salts made up of cations such as potassium, ammonium and the like and anions such as chloride, citrate, ascorbate, borate, phosphate, bicarbonate, sulfate, thiosulfate, bisulfate, sodium bisulfate, ammonium sulfate, and the like can also be used in addition to or instead of sodium chloride to achieve osmolalities within the above-stated range. Similarly, a sugar such as mannitol, dextrose, sorbitol, glucose and the like can also be used to adjust osmolality.

A particularly useful formulation of a composition of the invention provides achieving a sufficiently high tissue concentration with a minimum of doses so that a simple dosing regimen can be used to treat eye conditions with a catechin or polyphenol such as EGCG, as disclosed herein. In a particular embodiment, a depot of an a catechin or polyphenol such as EGCG composition is formed or supplied in contact with the external surface of the eye. A depot refers to a source of a composition containing a catechin or polyphenol such as EGCG that is not rapidly removed by tears or other eye clearance mechanisms. This allows for continued, sustained high concentrations of a catechin or polyphenol such as EGCG to be present in the fluid on the external surface of the eye by a single application. In general, it is believed that absorption and penetration are dependent on both the dissolved drug concentration and the contact duration of the external tissue with the drug-containing fluid. As the drug is removed by clearance of the ocular fluid and/or absorption into the eye tissue, more drug is provided, for example, dissolved, into the replenished ocular fluid from the depot.

The use of a depot can be used to facilitates loading of the ocular tissue with an ECGC composition. The depot can effectively slowly “pump” the composition containing a catechin or polyphenol such as EGCG into the ocular tissue. As the composition containing a catechin or polyphenol such as EGCG penetrates the ocular tissue, it is accumulated therein. As more composition containing a catechin or polyphenol such as EGCG is “pumped” in, the tissue concentration increases and the minimum inhibitory concentration threshold is eventually reached and/or exceeded, thereby loading the ocular tissue with a composition containing a catechin or polyphenol such as EGCG. By significantly exceeding the minimum inhibitory concentration (MIC) MIC₅₀, in particular the MIC₉₀ level, provided the toxicity limit is not exceeded, a therapeutically effective concentration will remain active in the tissue for an extended period of time. Thus, depending on the depot, one or two applications can provide a complete dosing regimen. Such a simple dosing regimen can provide a 6 to 14 day treatment concentration within the ocular tissue. A particularly useful dosing regimen involves one to two doses per day over a one to three day period, in partcicular one or two doses in a single day, to provide in vivo at least a 6 day treatment and more typically a 6 to 14 day treatment.

A depot can take a variety of forms so long as the composition containing a catechin or polyphenol such as EGCG can be provided in sufficient concentration levels therein and is releasable therefrom and that the depot is not readily removed from the eye. A depot generally remains for at least about 30 minutes after administration, in particular at least 2 hours and can be at least 4 hours. The term “remains” means that neither the depot composition nor the catechin or polyphenol such as EGCG is exhausted or cleared from the surface of the eye prior to the indicated time. In some embodiments, the depot can remain for up to eight hours or more. Typical ophthalmic depot forms include aqueous polymeric suspensions, ointments, and solid inserts. Polymeric suspensions are a particularly useful form.

Ointments are well known ophthalmic compositions and are essentially an oil-based delivery vehicle. Typical ointments use a petroleum and/or lanolin base to which is added the active ingredient and, optionally, excipients. Common bases include mineral oil, petrolatum and combinations thereof, but other oil bases can also be used. An ointment is usually applied as a ribbon onto the lower eyelid. The disadvantage of ointments is that they are difficult to administer, are messy, and uncomfortable/inconvenient to the patient; with temporarily blurred vision being common.

Inserts are another well known ophthalmic dosage form and are comprised of a matrix containing the active ingredient. The matrix is typically a polymer and the active ingredient is generally dispersed therein or bonded to the polymer matrix. The active ingredient is slowly released from the matrix through dissolution or hydrolysis of the covalent bond. In some embodiments, the polymer is bioerodible (soluble) and the dissolution rate thereof can control the release rate of the active ingredient dispersed therein. In another form, the polymer matrix is a biodegradable polymer that breaks down such as by hydrolysis to thereby release the active ingredient bonded thereto or dispersed therein. The matrix and active ingredient can be surrounded with a polymeric coating such as in the sandwich structure of matrix/matrix+active/matrix, to further control release as is well known in the art. The kinds of polymers suitable for use as a matrix are well known in the art. The composition containing a catechin or polyphenol such as EGCG can be dispersed into the matrix material or dispersed amongst the monomer composition used to make the matrix material prior to polymerization. The insert can be placed, depending on the location and the mechanism used to hold the insert in position, by either the patient or the doctor and is generally located under the upper eyelid. A variety of shapes and anchoring configurations, if any, are well known in the art. Generally, a biodegradable or bioerodible polymer matrix is used so that the spent insert does not have to be removed. As the biodegradable or bioerodible polymer is degraded or dissolved, the trapped composition containing a catechin or polyphenol such as EGCG is released. Although inserts can provide long term release and hence only a single application of the insert may be necessary, they are generally difficult to insert and are uncomfortable to the patient.

A particularly useful formulation is an aqueous polymeric suspension. The composition containing a catechin or polyphenol such as EGCG or the polymeric suspending agent is suspended in an aqueous medium. The composition containing a catechin or polyphenol such as EGCG can be in suspension although it is possible for the composition to be in solution (water soluble) or both in solution and in suspension in significant amounts. The polymeric suspending agent is generally a suspension, for example, water insoluble and/or water swellable, although water soluble suspending agents are also suitable for use with a suspension of the composition containing a catechin or polyphenol such as EGCG. The suspending agent serves to provide stability to the suspension and to increase the residence time of the dosage form on the eye. It can also enhance the sustained release of the drug in terms of both longer release times and a more uniform release curve.

Examples of polymeric suspending agents include dextrans, polyethylene glycols, polyvinylpyrolidone, polysaccharide gels, Gelrite™, cellulosic polymers like hydroxypropyl methylcellulose, and carboxy-containing polymers such as polymers or copolymers of acrylic acid, as well as other polymeric demulcents. A particularly useful polymeric suspending agent is a water swellable, water insoluble polymer, especially a crosslinked carboxy-containing polymer.

Crosslinked carboxy-containing polymers are well known in the art. In a particular embodiment, such polymers can be prepared from at least about 90% and in particular from about 95% to about 99.9% by weight, based on the total weight of monomers present, of one or more carboxy-containing monoethylenically unsaturated monomers, also referred to as carboxy-vinyl polymers. Acrylic acid is a particularly useful carboxy-containing monoethylenically unsaturated monomer, but other unsaturated, polymerizable carboxy-containing monomers, such as methacrylic acid, ethacrylic acid, β-methylacrylic acid (crotonic acid), cis-α-methylcrotonic acid (angelic acid), trans-α-methylcrotonic acid (tiglic acid), α-butylcrotonic acid, α-phenylacrylic acid, α-benzylacrylic acid, α-cyclohexylacrylic acid, β-phenylacrylic acid (cinnamic acid), coumaric acid (o-hydroxycinnamic acid), umbellic acid (p-hydroxycoumaric acid), and the like can be used in addition to or instead of acrylic acid.

Such polymers can be crosslinked by a polyfunctional crosslinking agent, preferably a difunctional crosslinking agent. The amount of crosslinking should be sufficient to form insoluble polymer particles, but not so great as to unduly interfere with sustained release of the composition containing a catechin or polyphenol such as EGCG. Typically the polymers are only lightly crosslinked. Particularly, the crosslinking agent is contained in an amount of from about 0.01% to about 5%, in particular from about 0.1% to about 5.0%, for example, from about 0.2% to about 1%, based on the total weight of monomers present. Included among such crosslinking agents are non-polyalkenyl polyether difunctional crosslinking monomers such as divinyl glycol; 2,3-dihydroxyhexa-1,5-diene; 2,5-dimethyl-1,5-hexadiene; divinylbenzene; N,N-diallylacrylamide; N,N-diallymethacrylamide and the like. Also included are polyalkenyl polyether crosslinking agents containing two or more alkenyl ether groupings per molecule, in particular alkenyl ether groupings containing terminal H₂C═C< groups, prepared by etherifying a polyhydric alcohol containing at least four carbon atoms and at least three hydroxyl groups with an alkenyl halide such as allyl bromide or the like, for example, polyallyl sucrose, polyallyl pentaerythritol, or the like (see, for example, Brown, U.S. Pat. No. 2,798,053, which is incorporated herein by reference). Diolefinic non-hydrophilic macromeric crosslinking agents having molecular weights of from about 400 to about 8,000, such as insoluble di-acrylates and polyacrylates and methacrylates of diols and polyols, diisocyanate-hydroxyalkyl acrylate or methacrylate reaction products of isocyanate terminated prepolymers derived from polyester diols, polyether diols or polysiloxane diols with hydroxyalkylmethacrylates, and the like, can also be used as the crosslinking agents (see, for example, Mueller et al., U.S. Pat. Nos. 4,192,827 and 4,136,250, each of which is incorporated herein by reference).

The crosslinked carboxy-vinyl polymers can be made from a carboxy-vinyl monomer or monomers as the sole monoethylenically unsaturated monomer present, together with a crosslinking agent or agents. In particular, the polymers are ones in which up to about 40%, and in particular from about 0% to about 20% by weight, of the carboxy-containing monoethylenically unsaturated monomer or monomers has been replaced by one or more non-carboxyl-containing monoethylenically unsaturated monomer or monomers containing only physiologically and ophthalmically innocuous substituents, including acrylic and methacrylic acid esters such as methyl methacrylate, ethyl acrylate, butyl acrylate, 2-ethylhexylacrylate, octyl methacrylate, 2-hydroxyethyl-methacrylate, 3-hydroxypropylacrylate, and the like, vinyl acetate, N-vinylpyrrolidone, and the like (see Mueller et al. U.S. Pat. No. 4,548,990, which is incorporated herein by reference).

Particularly useful polymers are lightly crosslinked acrylic acid polymers wherein the crosslinking monomer is 2,3-dihydroxyhexa-1,5-diene or 2,3-dimethylhexa-1,5-diene. Commercially available polymers include polycarbophil (Noveon™ AA-1) and Carbopol™. In particular, a carboxy-containing polymer system known by the tradename DuraSite™, containing polycarbophil, which is a sustained release topical ophthalmic delivery system that releases the drug at a controlled rate, is used in the aqueous polymeric suspension composition of the present invention.

The crosslinked carboxy-vinyl polymers used in practicing this invention are generally prepared by suspension or emulsion polymerizing the monomers, using conventional free radical polymerization catalysts, to a dry particle size of not more than about 50 μm in equivalent spherical diameter, for example, to provide dry polymer particles ranging in size from about 1 to about 30 μm, and in particular from about 3 to about 20 μm, in equivalent spherical diameter. Using polymer particles that were obtained by mechanically milling larger polymer particles to this size is generally avoided. In general, such polymers will have a molecular weight which has been variously reported as being from about 250,000 to about 4,000,000, and from 3,000,000,000 to 4,000,000,000.

In a particularly useful embodiment of the invention, the particles of crosslinked carboxy-vinyl polymer are monodisperse, meaning that they have a particle size distribution such that at least 80% of the particles fall within a 10 μm band of major particle size distribution. In particular, at least about 90% and, for example, at least about 95%, of the particles fall within a 10 μm band of major particle size distribution. Also, a monodisperse particle size means that there is no more than 20%, for example, no more than 10%, and in particular no more than 5% particles of a size below 1 μm. The use of a monodispersion of particles will give maximum viscosity and an increased eye residence time of the ophthalmic medicament delivery system for a given particle size. Monodisperse particles having a particle size of 30 μm and below are particularly useful. Good particle packing is aided by a narrow particle size distribution.

The aqueous polymeric suspension normally contains about 0.05 to about 1%, generally about 0.1 to about 0.5%, and particularly about 0.1 to about 0.5%, of the catechin or polyphenol such as EGCG and about 0.1 to about 10%, in particular about 0.5 to about 6.5% of a polymeric suspending agent. In the case of the above described water insoluble, water-swellable crosslinked carboxy-vinyl polymer, generally, an amount of the polymeric suspending agent is an amount ranging from about 0.5 to about 2.0%, in particular from about 0.5% to about 1.2%, and in certain embodiments from about 0.6 to about 0.9%, based on the weight of the composition. Although referred to in the singular, it is understood that one or more species of polymeric suspending agent such as the crosslinked carboxy-containing polymer can be used with the total amount falling within the stated ranges. In one particular embodiment, the composition contains about 0.6 to about 0.8% of a polycarbophil such as NOVEON™ AA-1.

In one embodiment, the amount of insoluble lightly crosslinked carboxy-vinyl polymer particles, the pH, and the osmotic pressure can be correlated with each other and with the degree of crosslinking to give a composition having a viscosity in the range of from about 500 to about 100,000 centipoise, and in particular from about 1,000 to about 30,000 or about 1,000 to about 10,000 centipoise, as measured at room temperature (about 25° C.) using a Brookfield Digital LVT Viscometer equipped with a number 25 spindle and a 13R small sample adapter at 12 rpm. Alternatively, when the viscosity is within the range of 500 to 3000 centipoise, it may be determined by a Brookfield Model DV-11+, choosing a number cp-52 spindle at 6 rpm. When water soluble polymers are used as the suspending agent, such as hydroxypropyl methylcellulose, the viscosity will typically be about 10 to about 400 centipoise, more typically about 10 to about 200 centipoise or about 10 to about 25 centipoise.

Aqueous polymeric suspensions of the present invention can be formulated so that they retain the same or substantially the same viscosity in the eye that they had prior to administration to the eye. Alternatively, they can be formulated so that there is increased gelation upon contact with tear fluid. For instance, when a formulation containing DuraSite™ or other similar polyacrylic acid-type polymer is administered to the eye at a pH of less than about 6.7, the polymer will swell upon contact with tear fluid since it has a higher pH (around 7). This gelation or increase in gelation leads to entrapment of the suspended particles, thereby extending the residence time of the composition containing a catechin or polyphenol such as EGCG in the eye. The catechin or polyphenol such as EGCG is released slowly as the suspended particles dissolve over time. All these events eventually lead to increased patient comfort and increased contact time of the composition containing a catechin or polyphenol such as EGCG with the eye tissues, thereby increasing the extent of drug absorption and duration of action of the formulation in the eye.

The viscous gels that result from fluid eye drops typically have residence times in the eye ranging from about 2 to about 12 hours, for example, from about 3 to about 6 hours. The agents contained in these drug delivery systems will be released from the gels at rates that depend on such factors as the drug itself and its physical form, the extent of drug loading and the pH of the system, as well as on any drug delivery adjuvants, such as ion exchange resins compatible with the ocular surface, which may also be present.

The compositions used to topically deliver the compositions of the present invention containing a catechin or polyphenol such as EGCG can be prepared from known or readily available materials through the application of known techniques by those skilled in the art. The compositions containing a catechin or polyphenol such as EGCG used in the present invention are commercially available or readily obtained by one skilled in the art using known techniques.

A composition of the invention containing a catechin or polyphenol such as EGCG is topically applied to an eye of a human or non-human animal, the latter including cows, sheep, horses, pigs, goats, rabbits, dogs, cats, and other mammals, for example, for veterinary uses. The composition can be applied as a liquid drop, ointment, a viscous solution or gel, a ribbon or as a solid. The composition can be topically applied, without limitation, to the front of the eye, under the upper eyelid, on the lower eyelid and/or in the cul-de-sac. The application can be as a treatment of an infection in the eye or as a preventive such as prior to surgery or post surgery.

Although discussed above as generally applying a composition of the invention containing a catechin or polyphenol such as EGCG topically to an eye, it is understood that other modes of administration can be used so long as the administration is effective for treating an ocular inflammation or ocular disease, as disclosed herein. For use as a therapeutic agent, a composition containing a catechin or polyphenol such as EGCG can be formulated with a pharmaceutically acceptable carrier to produce a pharmaceutical composition, which can be administered to the individual, which can be a human or other mammal, as discussed above. A pharmaceutically acceptable carrier can be, for example, water, sodium phosphate buffer, phosphate buffered saline, normal saline or Ringer's solution or other physiologically buffered saline, or other solvent or vehicle such as a glycol, glycerol, an oil such as olive oil or an injectable organic ester. The therapeutic compositions of the invention can also contain a carrier or excipient, many of which are known to one of ordinary skill in the art. Excipients that can be used include buffers, for example, citrate buffer, phosphate buffer, acetate buffer, and bicarbonate buffer; amino acids; urea; alcohols; ascorbic acid; glutathione; phospholipids; proteins, for example, serum albumin; ethylenediamine tetraacetic acid (EDTA); sodium chloride or other salts; liposomes; mannitol, sorbitol, glycerol, glucose, sucrose, dextrans; calcium or magnesium, and the like. The agents of the invention can be formulated in various ways, according to the corresponding route of administration. For example, liquid solutions can be made for ingestion or injection; gels or powders can be made for ingestion, inhalation, or topical application. Methods for making such formulations are well known and can be found in, for example, “Remington's Pharmaceutical Sciences,” 18th ed., Mack Publishing Company, Easton Pa. (1990).

A pharmaceutical composition containing a catechin or polyphenol such as EGCG can be administered to an individual by various routes, including by intravenous, subcutaneous, intramuscular, intrathecal or intraperitoneal injection; orally, as an aerosol spray; or by intubation. If desired, the composition can be incorporated into a liposome, a non-liposome lipid complex, or other polymer matrix, which further can have incorporated therein, for example, a second drug useful for treating the individual. Liposomes, which consist of phospholipids or other lipids, are nontoxic, physiologically acceptable and metabolizable carriers that are relatively simple to make and administer (Gregoriadis, Liposome Technology, Vol. 1 (CRC Press, Boca Raton Fla., 1984), which is incorporated herein by reference). The skilled artisan can readily determine an appropriate route and method of administration.

All of the percentages recited herein refer to weight percent, unless otherwise indicated. It is understood that modifications which do not substantially affect the activity the various embodiments of this invention are also provided within the definition of the invention provided herein. Accordingly, the following examples are intended to illustrate but not limit the present invention.

### EXAMPLE I

### Activity of EGCG Against Adenovirus In Vitro

This example describes the activity of EGCG against adenovirus in vitro.

EGCG is tested for activity against adenovirus in vitro. The activity of a catechin or polyphenol such as EGCG is tested essentially as described previously (Weber et al., Antiviral Res. 58:167-173 (2003), which is incorporated herein by reference). The effect of green tea catechins, and particularly the predominant component, epigallocatechin-3-gallate (EGCG), is tested on adenovirus infection in cell culture. Adding EGCG, for example, 100 microM, to the medium of infected cells is tested for reducing virus yield, IC₅₀ values and therapeutic index. The agents are tested for the most effective time to be added to the cells. The compounds are also tested for inhibitory activity of the viral protease adenain. In the studies described by Weber et al., a concentration of 0.005% was found to be effective in in vitro cultures. The results showed inhibitory effects and therapeutic index of >20.

Briefly, a suitable cell line, such as HepG2 cells, are grown in culture. Additional cell lines include, but are not limited to, HEK-293 and HeLa cells. The adenovirus is titered, for example, using plaque formation or end-point dilution. Experiments are generally performed at a multiplicity of infection (m.o.i.) of 10 plaque forming units (PFU) per cell. Two types of viral titers can be measured. Infectious titer refers to the number of adenovirus particles, which are fully developed and active. Particle titer refers to the number of total adenovirus, active and inactivated. Assays such as the plaque assay and the hexon assay measure the infectious titer. Assays such as ion exchange and reverse phase HPLC (RP-HPLC) and capillary electrophoresis (CE) measure total adenoviral particles, active and inactivated. RP-HPLC and CE can also be used to estimate the infectious titer.

For a viral plaque assay, a cell such as HepG2, HEK-293 or HeLa cells are grown in cell culture for the plaque assay. For example, HEK-293 cells are grown in cell culture media or medium plus 5-10% serum, such as calf serum, or proteins such as bovine serum albumin (BSA) or human serum albumin (HSA). Generally, about 500,000 cells per well are transferred into 6-well culture plates. After complete attachment over 24 hours, the media is removed and the wells are infected by various infectious medium (media plus virus or adenovirus). Such an assay can be performed, for example, as follows: well # 1: cell culture media only; well # 2: culture media plus undiluted viral sample; well # 3: culture media plus 10× diluted viral sample; well # 4: culture media plus 100× diluted viral sample; well # 5: culture media plus 1000× diluted viral sample; well # 6: culture media plus 10,000× diluted viral sample.

After 24 hours of incubation at 37° C. with 4-7% CO², all of the media is removed, and liquid agar made in cell culture media at 40-44° C. is added on the top of the cell layer. Plates are further incubated and the agar gelled at 37° C. The incubation is continued for 5-14 days, and the plates are observed every day and plaques are counted and marked from the bottom of the wells. Lack of bacterial contamination and continuous cell growth on well # 1 is considered a control that the result will be valid and therefore used for viral titer calculation. Wells 2-6 are counted and, if not too numerous to count (TNT), each plaque number is multiplied by the corresponding dilution factor and the average is calculated, which provides the initial viral load. The assay can be repeated with higher or lower dilution factors until the results become satisfactory.

Protocols and kits for titering adenovirus are also available from commercial sources. For example, the Adeno-X™ rapid titer kit can be used for titering advenovirus (Clontech, Mountain View Calif.). The Adenovirus Reference Material Working Group (ARMWG) provides information on standardization of adenovirus reference standards and characterization of samples (standard available from the American Type Culture Collection (ATCC), catalog No. VR-1516).

Catechins or polyphenols such as EGCG can be purified or extracted from green or black tea, synthesized, or obtained from commercial sources such as Sigma-Aldrich or LKT Laboratories. Alternatively, a green or black tea infusion can be used. The catechin or polyphenol is dissolved in a suitable buffer such as phosphate buffered saline (PBS) and diluted as needed for particular assay conditions. Various amounts of the catechin or polyphenol are tested for inhibitory activity against adenovirus for various times by adding to adenovirus infected cells. Inhibitory activity is measured, for example, by determining virus yield.

The catechin or polyphenol can also be tested by incubating virus in the presence of the catechin or polyphenol to test the ability of the catechin or polyphenol to inactivate virions. Purified virus is incubated with various concentrations of the catechin or polyphenol for a period of time, for example, 15 to 30 minutes, and then the incubated virus is diluted and added to cells to test for infectivity.

Other methods for testing the effect of catechins or polyphenols such as EGCG can also be used. For example, a hexon assay can be used to test the level of hexon production as an indication of adenovirus activity (see Bewig and Schmidt, BioTechniques 28:870-873 (2000), which is incorporated herein by reference). This assay uses an antibody against adenovirus hexon proteins, structural proteins in the adenovirus capsid, to visualize infected cells by immunocytochemistry staining. The assay is useful because it can be performed in a relatively short period of time, generally within a couple of days, as compared to the 5-14 days of the typical plaque assay, as discussed above. Concentrations of hexon in the infected cell cultures are directly correlated to the titer of the virus. Commercial kits are available to perform the hexon assay (see Cell Biolabs, San Diego Calif.; catalog No. VPK-109).

Other assays can be employed to determine the effect of catechins or polyphenols such as EGCG on adenovirus activity, including for example chromatography or capillary electrophoresis. Optionally, one or more purification steps can be included prior to such assays. A variety of purification methods are well known to those skilled in the art. One purification method uses simple sterile filtration. The sterile filtration can be performed using 0.22 μm (micron) filters (for small volume syringe filter) or by 0.1 μm filters. Filtration removes cells, debris and bacterial contaminants, providing relatively pure viral solutions. This technique can be sufficient for all required testing. Purification kits are available from a variety of commercial sources (for example, ViraKit™, Virapur, San Diego Calif.; Adeno-X™ virus purification kit, Clontech; Virabind™, Cell Biolabs; AdEasy™ virus purification kit, Stratagene, San Diego Calif.).

Additional purification methods are available to more efficiently remove proteins and incomplete viral particles. For example, density gradient centrifugation using cesium chloride or sucrose gradient purification can be used, in which the viral sample is centrifuged over a cesium or sucrose gradient. Cesium chloride is used most frequently. Other density gradient solutions include sodium iodide, sodium bromide, cesium sulfate, cesium acetate and potassium tartrate. For example, a cesium gradient is made in a centrifuge tube. Adenovirus sample is introduced on the top of the tube and centrifuged at 200,000 to 1,000,000 gram force for 10 minutes to one hour. The virus is collected by separation of the band formed in the tube. Viral particles that are not aggregated form a translucent band. Larger cell debris or aggregated virus go to the bottom of the tube and small particles and proteins stay on the very top. Density gradient methods for adenovirus purification are well known in the art (see, for example, Croyle et al., Pharm. Dev. Technol. 3:365-372 (1998), which is incorporated herein by reference).

Other methods can also be used to determine the effect of catechins or polyphenols such as EGCG on adenovirus. For example, an assay using ion exchange chromatography can be used to directly count the virus produced in the medium (see, for example, Transfiguracion et al., J. Chromatogr. B Biomed. Sci. Appl. 761:187-194 (2001), which is incorporated herein by reference). For example, anion exchange chromatography or HPLC can be used to quantify total adenovirus particles. A buffer such as 50 mM HEPES, pH 7.5, as described by Transfiguracion et al., or a similar buffer at around neutral pH can be used. A salt gradient such as 300 to 600 mM NaCl can be used to develop the anion exchange column and elute the bound virus. Such a method generally utilizes one or more purification steps prior to ion exchange chromatography, as discussed above.

In addition, reverse phase chromatography can be used (see, for example, Lehmberg et al., J. Chromatogr. B Biomed. Sci. Appl. 732:411-423 (1999), which is incorporated herein by reference). Such a method can be used to measure adenovirus particle concentration through quantification of structural proteins. During chromatography, intact adenovirus dissociates into its structural components (DNA and proteins) and the viral proteome is separated to yield a characteristic fingerprint. Components can be optionally identified using mass spectrometry, for example, matrix-associated laser desorption ionization time-of-flight mass spectrometry (MALDI-TOF-MS). The RP-HPLC is developed with a gradient of solvent A (0.1% trifluoroacetic acid (TFA) in water) and solvent B (0.1% TFA in acetonitrile), generally a gradient of 20% solvent B to 60% solvent B. It is possible to evaluate the viral titer, both active and inactive, by reverse phase HPLC (RP-HPLC) but, due to the nature of RP-HPLC, which shows all of the proteins present in the sample, the sample is generally purified before titer evaluation. The purification can utilize any of the well known purification methods, including but not limited to those described above, for example, ultrafiltration, density gradient centrifugation using a sucrose or cesium gradient, ion exchange chromatography, for example, ion exchange HPLC, or use of a commercially available purification kit.

Other methods can also be used to determine the effect of catechins or polyphenols such as EGCG on adenovirus. For example, capillary electrophoresis (CE) can be used (see, for example, Mann et al., J. Chromatogr. A 895:329-337 (2000), which is incorporated herein by reference). The CE can be performed in 25 mM sodium phosphate, pH 7.0, as electrophoresis buffer. Generally, one or more optional purification steps, including but not limited to those discussed above, are used prior to CE due to the potential interference of cell culture medium, animal tissue or human tissue related impurities so that debris and non-viral particles are removed prior to CE. The purification for CE can be as simple as an ultrafiltration step since, unlike RP-HPLC, the adenovirus will not be denatured during a CE run. Thus, 0.22 μm filtration can be sufficient for a CE test. The filtration will remove all large particles, and adenovirus can be separated from other impurities as part of the CE assay procedure.

### EXAMPLE II

### Animal Model for Treating Adenovirus Ocular Infection

This example describes animal models suitable for EGCG, other compounds, or combinations of EGCG with other antimicrobial agents.

Adenovirus is inoculated intrastromally or topically. A non-replicative lacZ adenovirus can be used for a prophylaxis indication to study the mechanism of adenovirus infection. An exemplary animal model for ocular infection is described in Kaneko et al., Antiviral Res. 61:63-66 (2004), which is incorporated herein by reference.

To determine the antiviral effects of compounds against ocular adenovirus (AdV) infection, an established animal model of adenovirus infection such as cotton rat eyes is used (see Kaneko et al., supra, 2004). Briefly, cotton rat eyes are inoculated intrastromally and topically with adenovirus, for example, any one or combination of the adenovirus serotypes, and treated topically with test compounds in eye drops, for example, twice a day. The infected corneas are extracted and homogenized, and virus titers in the cornea specimens are determined by a plaque assay. Adenovirus antigen in the infected corneas is also determined in the corneal epithelial cells by immunofluorescence stain. Mean virus titer and virus shedding duration is compared with those of a control group.

In more detail, virus is inoculated into the bilateral eyes of 10-week old female cotton rats or other suitable age and gender matched rats are used. The cotton rats are anesthetized with a peritoneal injection of sodium pentobarbital and topical 0.4% oxybuprocaine hydrochloride drops added to each eye, or other suitable anesthetics are used. A suitable volume, for example, 30 microliters containing adenovirus, such as 4×10⁵ pfu/ml or other suitable virus concentrations, are inoculated into the central cornea intrastromally with a 30 G needle to form two focal blebs (dice pattern). The cornea is then scarified with a 27 G needle superficially and inoculated topically with another volume, such as 20 microliters, of virus suspension. The lids are closed and the eye massaged through the lids for 30 seconds. Groups of rats can be inoculated with various adenovirus types, including adenovirus 4, 5, 8 or 37, other adenovirus types, as desired. Following viral adsorption for a suitable period of time, such as 2 hours, all inoculated eyes are irrigated with a balanced salt solution to wash out unadsorbed virus.

Virus titer in the cornea is estimated on various days, including day 0. The cornea is cut circularly along with the limbus, extracted with surgical scissors for an eye operation, and placed in a suitable buffer such as Eagle's minimum essential medium (MEM), for example, a 0.2 ml volume. After homogenization for 30 seconds on ice, the specimens are centrifuged at 3000 rpm for 10 minutes and virus in the supernatant is titrated on an appropriate cell line such as A549 cells by plaque assay. Alternatively, virus titers can also be determined by swabbing the upper and lower fornices of each eye with a cotton applicator and placed in a volume of buffer such as MEM for titering on a suitable cell line.

LacZ adenovirus (replicative) is used to infect the test animal eyes. The treated eyes are compared with the untreated eyes at 1 to 7 days, every day. The corneas can be stained for LacZ expression and the degree of stain evaluated. The staining procedure is used to confirm the infection and the treatment as an endpoint.

### EXAMPLE III

### Testing of Anti-Adenoviral Formulations

This example describes the testing of various formulations of EGCG for anti-adenoviral activity alone or in combination with other antimicrobial agents.

The efficacy of various formulations of EGCG is tested using human embryonic kidney 293 (HEK 293) cells in plaque assays and viral replication assays. Synergistic activity is tested with combinations of EGCG and one or more antimcirobial agents or antibiotics, including antiviral agents.

Briefly, HEK 293 cell layers are prepared by cell culture in several 6-well cell culture plates. The cells are incubated at 37° C. with 4-6% CO₂ and required humidity to form a complete layer. The media is removed and replaced with fresh media. Viral particles are added with increasing concentrations in five wells and one well is reserved as a control. The plates are incubated overnight and the media is removed the next day. The cells are covered with Agar/growth media solution at the concentration of Agar to form a gel layer to limit the cell movement and prevent disruption of the cell layer. One 6-well plate is saved as a control while the rest are used to add the test compounds or their combinations. Each day, starting at the second day the “plaques” in cell layers of each well are counted and documented. The results are summarized after two weeks and the plates with less plaques indicate the effectiveness of the compound.

### EXAMPLE IV

### Exemplary Formulations

This example describes various formulations containing EGCG. It is understood that these, variations on these formulations and other formulations can be used.

Formulation 1: Preservative-Free Formulation
EGCG (0.5%)
Azithromycin (1.0%)
Mannitol (1%)
NaCl (0.5%)
Citric buffer
Water to dissolve
NaOH to adjust pH to 6.3

Formulation 2: Preservative-Free Formulation
EGCG (0.5%)
Azithromycin (1.0%)
Citric buffer
Mannitol (1%)
Poloxamer (0.5% Pluronic or Lutrol F-68 or F-127)
Water to dissolve
NaOH to adjust pH to 6.3

Formulation 3: Preservative-Free Formulation
EGCG (0.5%)
Azithromycin (1.0%)
Noveon™ AA-1 (polycarbophil) (1.0%)
Sodium Chloride (0.5%)
Mannitol (1%)
Ethylenediamine tetraacetic acid (EDTA) (0.1%)
Citric buffer
Water to dissolve
NaOH to adjust pH to 6.3

Formulation 4: Preservative-Free Formulation
EGCG (0.5%)
Azithromycin (1.0%)
Citric buffer
Noveon™ AA-1 (polycarbophil) (1.0%)
Sodium Chloride (0.45%)
Mannitol (1%)
EDTA (0.1%)
Poloxamer (0.5% Pluronic or Lutrol F-68 or F-127)
Water to dissolve
NaOH to adjust pH to 6.3

Additional formulations with preservative: the same as Formulations 1-4 with the addition of 0.001 to 0.02% Benzalkonium Chloride, for example, 0.001%, 0.002%, 0.003%, 0.004%, 0.005%, 0.0075%, 0.01%. 0.0125%, 0.015%, 0.0175%, 0.02%, and the like.

Additional formulations with preservative: the same as Formulations 1-4 with the addition of 0.5% to 5% of potassium sorbate, for example, 0.05%, 0.1%, 0.2%, 0.3%, 0.4%, 0.5%, 1%, 2%, 5%, and the like.

Throughout this application various publications have been referenced. The disclosures of these publications in their entireties are hereby incorporated by reference in this application in order to more fully describe the state of the art to which this invention pertains. Although the invention has been described with reference to the examples provided above, it should be understood that various modifications can be made without departing from the spirit of the invention.

What is claimed is:

1. A method of treating an ocular infection, comprising administering to an eye of a patient having an ocular infection an effective amount of a catechin or polyphenol.

2. The method of claim 1, wherein said catechin or polyphenol is (−)-epigallocatechin gallate (EGCG).

3. The method of claim 1, wherein said catechin or polyphenol is selected from epicatechin (EC), epicatechin gallate (ECG), epigallocatechin (EGC), gallocatechin gallate (GCG), apigenin, anthocyanin, aurone, chalcone, isoflavone, proanthocyanidin, astringin, coumarin, stilbene and xanthone.

4. The method of claim 1, wherein said ocular infection is a viral infection.

5. The method of claim 4, wherein said viral infection is an adenoviral infection.

6. The method of claim 1, wherein said catechin or polyphenol is administered as a pharmaceutical composition as a solution, suspension or ointment.

7. The method of claim 1, wherein said catechin or polyphenol is administered with an antimicrobial agent.

8. The method of claim 1, wherein said antimicrobial agent is selected from an antiviral agent, an antibacterial agent, or an anti-fungal agent.

9. The method of claim 1, wherein said catechin or polyphenol is administered with a penetration-enhancing agent.

10. A method of ameliorating a sign or symptom associated with an ocular infection, comprising administering to an eye of a patient having an ocular infection an effective amount of a catechin or polyphenol.

11. The method of claim 10, wherein said catechin or polyphenol is (−)-epigallocatechin gallate (EGCG).

12. The method of claim 10, wherein said catechin or polyphenol is selected from epicatechin (EC), epicatechin gallate (ECG), epigallocatechin (EGC), gallocatechin gallate (GCG), apigenin, anthocyanin, aurone, chalcone, isoflavone, proanthocyanidin, astringin, coumarin, stilbene and xanthone.

13. The method of claim 10, wherein said ocular infection is a viral infection.

14. The method of claim 13, wherein said viral infection is an adenoviral infection.

15. The method of claim 10, wherein said catechin or polyphenol is administered as a pharmaceutical composition as a solution, suspension or ointment.

16. The method of claim 10, wherein said catechin or polyphenol is administered with an antimicrobial agent.

17. The method of claim 10, wherein said antimicrobial agent is selected from an antiviral agent, an antibacterial agent, or an anti-fungal agent.

18. The method of claim 10, wherein said catechin or polyphenol is administered with a penetration-enhancing agent.

19. A method of treating ocular inflammation, comprising administering to an eye of a patient having ocular inflammation an effective amount of a catechin or polyphenol.

20. The method of claim 19, wherein said catechin or polyphenol is (−)-epigallocatechin gallate (EGCG).

21. The method of claim 19, wherein said catechin or polyphenol is selected from epicatechin (EC), epicatechin gallate (ECG), epigallocatechin (EGC), gallocatechin gallate (GCG), apigenin, anthocyanin, aurone, chalcone, isoflavone, proanthocyanidin, astringin, coumarin, stilbene and xanthone.

22. The method of claim 19, wherein said catechin or polyphenol is administered as a pharmaceutical composition as a solution, suspension or ointment.

23. The method of claim 19, wherein said catechin or polyphenol is administered with a penetration-enhancing agent.

24. A method of ameliorating a sign or symptom associated with ocular inflammation, comprising administering to an eye of a patient having ocular inflammation an effective amount of catechin or polyphenol

25. The method of claim 24, wherein said catechin or polyphenol is (−)-epigallocatechin gallate (EGCG).

26. The method of claim 24, wherein said catechin or polyphenol is selected from epicatechin (EC), epicatechin gallate (ECG), epigallocatechin (EGC), gallocatechin gallate (GCG), apigenin, anthocyanin, aurone, chalcone, isoflavone, proanthocyanidin, astringin, coumarin, stilbene and xanthone.

27. The method of claim 24, wherein said catechin or polyphenol is administered as a pharmaceutical composition as a solution, suspension or ointment.

28. The method of claim 24, wherein said catechin or polyphenol is administered with a penetration-enhancing agent.

29. A method of treating an ocular cancer, comprising administering to an eye of a patient having an ocular cancer an effective amount of a catechin or polyphenol (−)-epigallocatechin gallate (EGCG).

30. The method of claim 29, wherein said catechin or polyphenol is (−)-epigallocatechin gallate (EGCG).

31. The method of claim 29, wherein said catechin or polyphenol is selected from epicatechin (EC), epicatechin gallate (ECG), epigallocatechin (EGC), gallocatechin gallate (GCG), apigenin, anthocyanin, aurone, chalcone, isoflavone, proanthocyanidin, astringin, coumarin, stilbene and xanthone.

32. The method of claim 29, wherein said catechin or polyphenol is administered as a pharmaceutical composition as a solution, suspension or ointment.

33. The method of claim 29, wherein said catechin or polyphenol is administered with a chemotherapeutic agent.

34. The method of claim 29, wherein said catechin or polyphenol is administered with a penetration-enhancing agent.

35. A method of treating a benign eye tumor, comprising administering to an eye of a patient having a benign eye tumor an effective amount of a catechin or polyphenol.

36. The method of claim 35, wherein said catechin or polyphenol is (−)-epigallocatechin gallate (EGCG).

37. The method of claim 35, wherein said catechin or polyphenol is selected from epicatechin (EC), epicatechin gallate (ECG), epigallocatechin (EGC), gallocatechin gallate (GCG), apigenin, anthocyanin, aurone, chalcone, isoflavone, proanthocyanidin, astringin, coumarin, stilbene and xanthone.

38. The method of claim 35, wherein said catechin or polyphenol is administered as a pharmaceutical composition as a solution, suspension or ointment.

39. The method of claim 35, wherein said catechin or polyphenol is administered with a chemotherapeutic agent.

40. The method of claim 35, wherein said catechin or polyphenol is administered with a penetration-enhancing agent.

41. A pharmaceutical composition comprising a catechin or polyphenol and a pharmaceutically acceptable carrier.

42. The pharmaceutical composition of claim 41, wherein said catechin or polyphenol is (−)-epigallocatechin gallate (EGCG).

43. The pharmaceutical composition of claim 41, wherein said catechin or polyphenol is selected from epicatechin (EC), epicatechin gallate (ECG), epigallocatechin (EGC), gallocatechin gallate (GCG), apigenin, anthocyanin, aurone, chalcone, isoflavone, proanthocyanidin, astringin, coumarin, stilbene and xanthone.

44. The pharmaceutical composition of claim 41, wherein said pharmaceutical composition is a solution, suspension or ointment.

45. The pharmaceutical composition of claim 41, wherein said pharmaceutical composition is formulated for ophthalmic administration.

46. The pharmaceutical composition of claim 41, wherein is formulated to be administered by injection.

47. The pharmaceutical composition of claim 41, wherein said catechin or polyphenol is administered with an antimicrobial agent.

48. The pharmaceutical composition of claim 41, wherein said antimicrobial agent is selected from an antiviral agent, an antibacterial agent, or an anti-fungal agent.

49. The pharmaceutical composition of claim 41, wherein said catechin or polyphenol is administered with a chemotherapeutic agent.

50. A composition comprising 0.5% (−)-epigallocatechin gallate (EGCG), 1% azithromycin, 0.5% NaCl and citric buffer, pH 6.3.

51. The composition of claim 50, further comprising 0.5% poloxamer.

52. The composition of claim 50, further comprising 1% polycarbophil, 0.45% sodium chloride, 1.0% manntiol and 0.1% ethylenediamine tetraacetic acid (EDTA).

53. The composition of claim 50, further comprising 1% polycarbophil, 0.45% sodium chloride, 1.0% mannitol, 0.1% EDTA and 0.5% poloxamer.

54. The composition of claim 50, further comprising 0.001 to 0.02% benzalkonium chloride.

55. The composition of claim 50, further comprising 0.05% to 5% potassium sorbate.
